(19) United States
(12) Patent Application Publication
(10) Pub. No.: US 2002/0111580 A1
(43) Pub. Date: Aug. 15, 2002

(54) Drug dispenser

(76) Inventors: Rodger J. Richeal, Campbell, CA (US); Jeffrey Y. Chen, Mountain View, CA (US); Mark D. Senatori, Palo Alto, CA (US); Ray McKinnon, Castro Valley, CA (US); Wai Loong-Lim, Palo Alto, CA (US)

Correspondence Address: MORRISON & FOERSTER LLP, 755 PAGE MILL RD, PALO ALTO, CA, 94304-1018, US

(21) Appl. No.: 09/942,795

(22) Filed: Aug. 29, 2001

Related U.S. Application Data
(63) Continuation of application No. 09/106,805, filed on Jun. 29, 1998, now abandoned.

(51) Int. Cl. A61M 37/00

(52) U.S. Cl. 604/89

(57) ABSTRACT

A drug dispenser for administering a drug solution ophthalmically. The dispenser comprises a cylindrical member having an open proximal end, a distal end having a hole for dispensing the drug solution, and a connecting passage formed in a sidewall thereof. A plunger is receivable in the proximal end of the cylindrical member and the member is axially slidable on the plunger between a non-mix position and a mix position. The dispenser further comprises a piston having a distal end and a proximal end and slidably disposed within the cylindrical member in sealing engagement therewith. The distal end of the piston is located proximally of the passage and forms a drug chamber with the distal end of the member for storing a drug when the member is in its non-mix position. The proximal end of the piston is distally spaced from the plunger to form a diluent chamber therebetween for storing a diluent when the cylindrical member is in its non-mix position. When the cylindrical member is moved to its mix position the drug chamber and diluent chamber are in fluid communication and the diluent is free to flow through the passage to mix with the drug to form the drug solution.

### TECHNICAL FIELD OF THE INVENTION

This invention is in the field of drug dispensers. More particularly, it relates to a hand-held drug dispenser which stores a liquid diluent and a drug in separate chambers and is manipulated to reconstitute the drug just prior to administration.

### BACKGROUND OF THE INVENTION

A solution of drug or similar therapeutic agent must be of precisely known concentration to avoid over or under treatment. In addition, the drug must be stable over time to avoid the problems of reduced dosage of active ingredients and the possible side effects of the decomposition products of the drug. One well known approach to this problem is to keep the drug in a dry form, such as a tablet or capsule, or in a solid form, such as a powder or lyophilized mass, that can be mixed with water or other appropriate solvent and reconstituted. Another problem that occurs in drug delivery systems includes the sensitivity of some drugs to the highly acidic conditions in the stomach or digestinal enzymes in the gastrointestinal tract that may destroy the drug. Such drugs cannot generally be delivered in a tablet or capsule form. An alternative method for delivery of drugs involves direct injections of a drug solution into the blood stream. This method however, requires trained personnel to administer the drug due to possible problems with direct injections and the handling of syringes and needles. For these reasons there is increased interest in drug delivery systems that involve delivery by inhalation or by application of eye drops. Since ease of use is one benefit of such a drug delivery system, it is necessary that the methods used to stabilize the drug and reconstitute the drug solution with high quantitative accuracy be as simple as possible.

The present invention provides an ophthalmic drug dispenser that stores precise quantities of diluent and drug in a segregated manner and is easily manipulated to reconstitute the drug and deliver it dropwise into an eye.

### DISCLOSURE OF THE INVENTION

The invention is a drug dispenser for administering a drug solution ophthalmically. The dispenser generally comprises a cylindrical member having an open proximal end, a distal end having a hole formed therein for dispensing the drug solution, and a connecting passage formed in a sidewall thereof. A plunger is receivable in the proximal end of the cylindrical member and the member is axially slidable on the plunger between a non-mix position and a mix position. A piston having a distal end and a proximal end is slidably disposed within the cylindrical member in sealing engagement therewith. The distal end of the piston is located proximally of the connecting passage and forms a drug chamber with the distal end of the cylindrical member for storing a drug when the cylindrical member is in its non-mix position. The proximal end of the piston is distally spaced from the plunger to form a diluent chamber therebetween for storing a physiologically acceptable liquid diluent when the cylindrical member is in its non-mix position. When the cylindrical member is moved to its mix position the drug chamber and diluent chamber are in fluid communication and the diluent is free to flow through the connecting passage to mix with the drug to form the drug solution.

In another aspect of the invention, a drug dispenser generally comprises a sealed diluent chamber containing a physiologically acceptable liquid diluent and a sealed drug chamber containing a drug. At least a portion of both chambers is flexible. The dispenser further comprises a housing containing the diluent chamber and the drug chamber. The housing has an opening at a distal end thereof for delivery of the drug solution. A piston is axially movable within the housing to open the sealed chambers and allow the drug and diluent to mix to form the drug solution.

In yet another aspect of the invention a drug dispenser is movable between a non-mix position wherein the drug and diluent are separated and a mix position wherein the drug and diluent are mixed to form the drug solution. The drug dispenser comprises a piston and an actuator assembly for delivering the drug when the dispenser is in its mix position. The actuator assembly comprises a drive mechanism operable to move the piston in a distal direction and a ratchet gear for preventing movement of the piston in a proximal direction.

The above is a brief description of some deficiencies in the prior art and advantages of the present invention. Other features, advantages, and embodiments of the invention will be apparent to those skilled in the art from the following description, drawings, and claims.

### BRIEF DESCRIPTION OF THE DRAWINGS

FIG. 1 is a perspective of a drug dispenser of the present invention;

FIG. 2 is an exploded view of the drug dispenser of FIG. 1;

FIG. 3 is a schematic cross-sectional view of the drug dispenser of FIG. 1 in a non-mix position;

FIG. 4 is a schematic cross-sectional view of the drug dispenser of FIG. 1 in a mix-position;

FIG. 5 is a schematic cross-sectional view of the drug dispenser of FIG. 4 with a cap removed;

FIG. 6A is a cross-sectional view of a cylindrical member of the drug dispenser of FIG. 1;

FIG. 6B is a perspective of a gear of the drug dispenser of FIG. 1;

FIG. 6C is a top view of an actuator button of the drug dispenser of FIG. 1;

FIG. 7 is a second embodiment of a drug dispenser of the present invention;

FIG. 8A is a bag of the drug dispenser of FIG. 7;

FIG. 8B is the bag of FIG. 8A with one end sealed;

FIG. 8C is the bag of FIG. 8B with diluent therein;

FIG. 8D is the bag of FIG. 8C with a second seal forming a sealed diluent chamber in the bag;

FIG. 8E is the bag of FIG. 8D with a drug therein;

FIG. 8F is the bag of FIG. 8E with a third seal forming a sealed drug chamber;

FIG. 8G is the bag of FIG. 8F with a nozzle attached thereto;

FIG. 9 is a third embodiment of a drug dispenser of the present invention;

FIG. 10A is an empty drug chamber of the drug dispenser of FIG. 9;

FIG. 10B is the drug chamber of FIG. 10A filled with a drug;

FIG. 10C is the drug chamber of FIG. 10B with a sheet sealing the chamber;

FIG. 11A is an empty diluent chamber of the drug dispenser of FIG. 9;

FIG. 11B is the diluent chamber of FIG. 11A filled with a diluent; and

FIG. 11C is the diluent chamber of FIG. 11B with a sheet sealing the chamber.

Corresponding reference characters indicate corresponding parts throughout the several views of the drawings.

### MODES FOR CARRYING OUT THE INVENTION

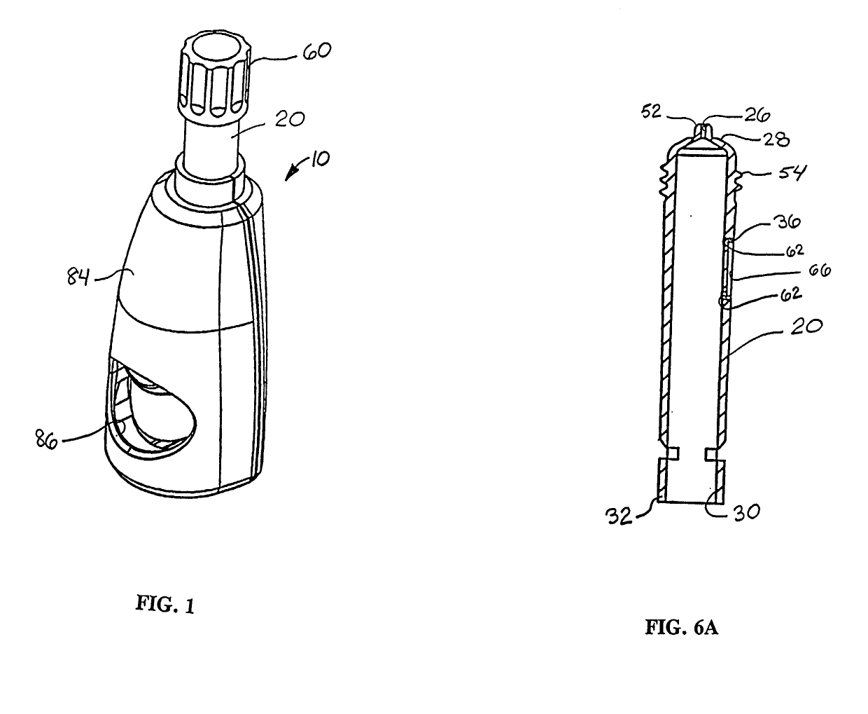

Referring now the drawings, and first to FIG. 1, a drug dispenser of the present invention is generally indicated at 10. The drug dispenser 10 is used for administering a drug solution to a person. The drug solution is preferably formed from a lyophilized drug and a physiologically acceptable liquid diluent, and is preferably administered ophthalmically. The dispenser 10 is designed to store the lyophilized drug separately from the liquid diluent until just prior to use when the drug is mixed with the diluent to form the drug solution.

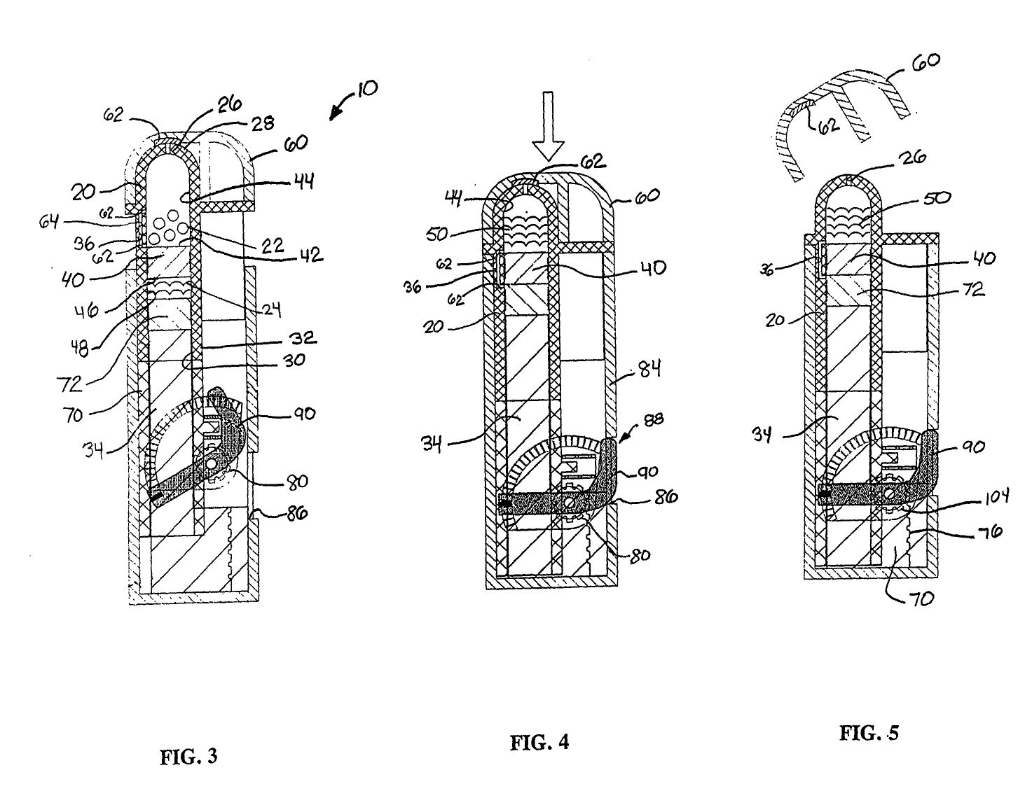

The dispenser 10 includes a cylindrical member 20 for storing and mixing a drug 22 and diluent 24 (FIG. 3). The cylindrical member 20 has an opening 26 at its distal end 28 for delivering the drug solution, an opening 30 at its proximal end 32 for receiving a plunger 34, and a connecting passage 36 formed in a sidewall of the member for mixing the diluent 24 with the drug 22. The cylindrical member 20 is axially slidable on the plunger 34 between a non-mix position (FIG. 3) and a mix position (FIG. 4). A piston 40 is slidably disposed within the cylindrical member 20. When the cylindrical member 20 is in its non-mix position, a distal end 42 of the piston 40 is located proximally of the connecting passage 36 to form a drug chamber 44 with the distal end 28 of the cylindrical member 20 for storing the drug 22 (FIG. 3). A proximal end 46 of the piston 40 is distally spaced from the plunger 34 to form a diluent chamber 48 therebetween for storing the diluent 24 when the cylindrical member 20 is in its non-mix position. When the cylindrical member 20 is moved to its mix position, the drug chamber 44 and diluent chamber 48 are in fluid communication and the diluent 24 is free to flow through the connecting passage 36 to mix with the drug 22 to form the drug solution 50 (FIGS. 3 and 4).

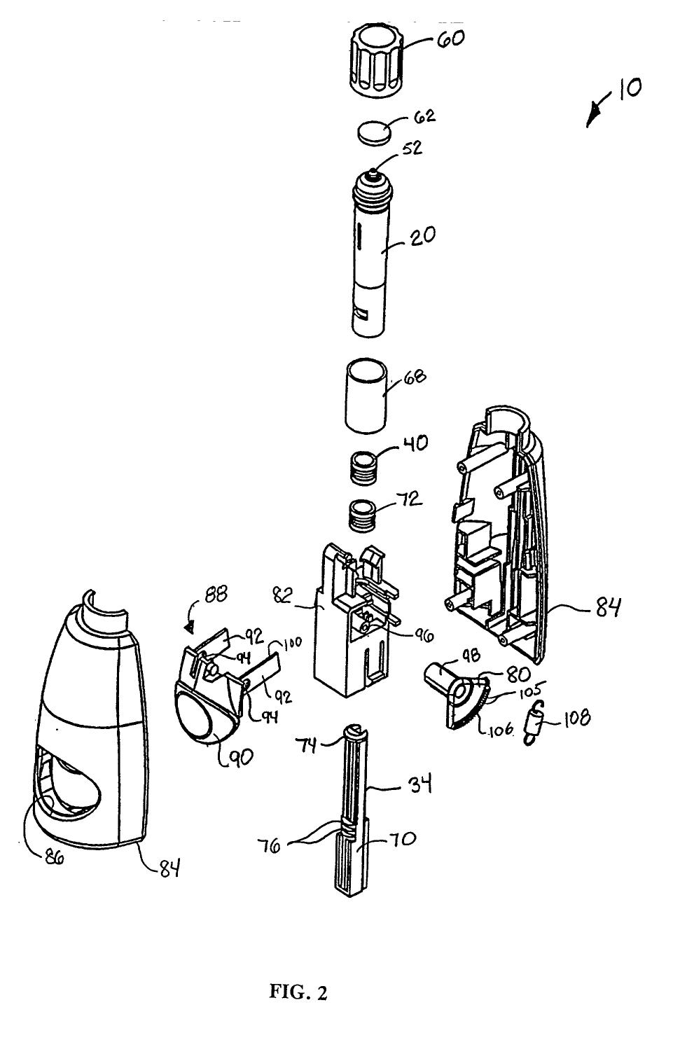

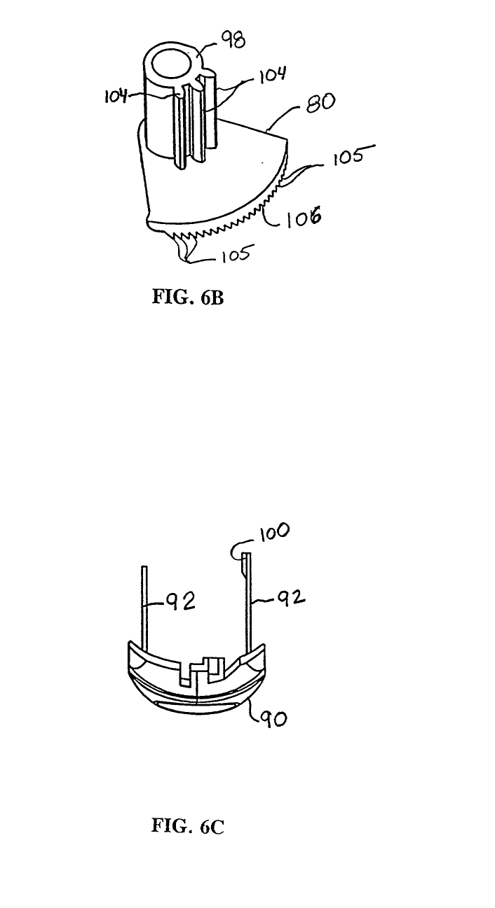

The connecting passage 36 is formed in the sidewall of the cylindrical member 20 and comprises two radial holes 62 and a longitudinal connecting slot 64 located between the two holes (FIG. 3). The slot 64 may be replaced with a longitudinal groove 66 in the outer wall of the cylindrical member 20 which allows the diluent 24 to pass between the outer wall of the member and a sleeve 68 surrounding the cylindrical member (FIGS. 2 and 6A). A groove (not shown) may also be formed in the inner wall of the cylindrical member 20 to allow the diluent 24 to pass between the piston 40 and the inner wall of the member. The connecting passage 36 is sized to prevent passage of the drug 22 through the passage and minimize the fluid volume of the passage to limit the amount of drug solution 50 remaining in the passage when the cylindrical member 20 is in its mix position (FIG. 4).

As shown in FIG. 6A, a nozzle 52 is formed at the distal end 28 of the cylindrical member 20 for delivery of the drug solution 50. The length and diameter of the nozzle 52 are preferably minimized to limit the amount of drug solution 50 located within the nozzle 52 when the cap 60 is removed (FIG. 5). The distal end 28 of the cylindrical member 20 is preferably generally spherical shaped to provide a constant drop volume regardless of the orientation of the drug dispenser 10 during delivery of the drug solution. The nozzle 52 has a diameter ranging from about 0.080 inches to 0.250 inches, for example. The cylindrical member 20 further includes threads 54 formed in the outer wall near the distal end 28 of the member. The external threads 54 are provided for engagement with internal threads located in a cap 60 of the dispenser 10. The cap 60 prevents contamination of the nozzle 52 or possible entry of contaminants, moisture, or humidity into the drug chamber 44. The cap 60 preferably includes an elastomeric seal 62 on the inside surface of the cap to seal the nozzle 52 (FIGS. 2 and 3). The cap 60 may also be retained on the distal end 28 of the cylindrical member 20 by a snap fit, detent, or any other suitable methods. The cylindrical member 20 may have a height of 2.5 inches, an outer diameter of 0.42 inch and an inner diameter of 0.33 inch, for example. The cylindrical member 20 may be formed from a polymeric material such as polypropylene, or any other suitable material. The material of the cylindrical member 20 as well as the material of the other internal components of the dispenser 10 should be selected for compatibility with the drug 22, diluent 24, and drug solution 50. It is to be understood that the cylindrical member 20 may have a shape or dimensions different than those disclosed herein without departing from the scope of the invention.

The piston 40 is axially slidable within the cylindrical member 20 and has an outer diameter slightly larger than the inner diameter of the cylindrical member prior to insertion into the member, so that the piston sealingly engages the inner wall of the member to prevent leakage of the diluent 24 between the piston and the inner wall of the member. The length of the piston 40 is slightly smaller than the distance between the holes 62 of the connecting passage 36 so that when the piston is in its mix position it can be positioned between the two holes to open up the passage and allow fluid communication between the drug chamber 44 and the diluent chamber 48 (FIG. 4). The piston 40 may be formed from a rubber material or any other suitable material which is compatible with the material of the cylindrical member 20, drug 22, and diluent 24.

The plunger 34 is partially disposed within the cylindrical member 20 and is located proximally of the piston 40 (FIG. 3). The plunger 34 comprises a pushrod 70 and a sealing piston 72 which may be the same as the piston 40 described above (FIG. 2). The sealing piston 72 may also be integrally formed with the pushrod 70. The pushrod 70 is generally rectangular in shape and has a generally cylindrical edge margin 74 at its distal end for sliding engagement with the inner wall of the cylindrical member 20. The pushrod 70 includes teeth 76 located along one side of the pushrod at generally a middle portion of the pushrod. The teeth 76 are provided for engagement with a gear 80 for movement of the plunger 34 in the distal direction as further described below. The number, arrangement, and shape of teeth 76 may be different than shown without departing from the scope of the invention.

The cylindrical member 20 and pushrod 70 are mounted within a support 82 which coaxially aligns the cylindrical member and pushrod. The support 82, pushrod 70, and a portion of the cylindrical member 20 are contained within a housing 84. The housing 84 includes an opening (window) 86 for access to an actuator assembly, generally indicated at 88, which is used to deliver the drug solution 50 after the drug 22 and diluent 24 are mixed. The housing 84 is preferably formed of two separate pieces which snap fit together. The pieces of the housing may also be glued together, or attached by any other suitable means. The housing 84 may be formed from a polymeric material such as ABS, or any other suitable material.

The pushrod 70 is initially positioned at the proximal end of the housing 84 with the support 82 and cylindrical member 20 spaced from the proximal end of the housing 84. The cylindrical member 20 and support 82 move proximally along the plunger 34, which remains stationary, when the cylindrical member is moved from its non-mix to its mix position. The plunger 34 only moves relative to the housing during delivery of the drug solution 50 after the drug 22 and diluent 24 are mixed (FIG. 5).

The actuator assembly 88 includes an actuation button 90 which is positioned within the window 86 in the housing 84 when the cylindrical member 20 is in its mix position (FIG. 4). When the cylindrical member 20 is in its non-mix position, the button 90, which is connected to the support 82, is positioned above the window 86 so that the button can not inadvertently be actuated prior to the mixing of the drug 22 and diluent 24 (FIGS. 1 and 3). When the cylindrical member 20 is in its mix-position and the button 90 is positioned within the window 86, the button is actuated by applying an inwardly directed radial force on the button (FIG. 4). The button 90 preferably requires only about 2.0 in-lbf force for full actuation. The actuator assembly 88 further includes two arms 92 extending inwardly from the button 90 (FIG. 2). Each arm 92 has an opening 94 formed therein for receiving a shaft 96 connected to the support 82 for pivotally mounting the button 90 on the support. The shaft 96 extends through the support 82 and is fixedly mounted thereon. The actuator arms 92 are pivotally connected to the shaft 96.

The actuator assembly 88 further includes gear 80. The gear 80 comprises a tubular member 98 which slides over the end of the shaft 96 and a plurality of teeth 104 extending radially outward from the tubular member and configured for intermeshing engagement with the teeth 76 of the pushrod 70 (FIGS. 2 and 6B). The gear 80 includes a ratchet gear 106 having teeth 105 configured for engagement with a rib 100 extending from an inner surface of one of the actuator arms 92 for rotation of the gear by the actuation button 90 (FIGS. 2 and 6C). The teeth 105 of the ratchet gear 106 are preferably designed to permit only one-way movement of the gear to prevent the pushrod 70 from moving in the proximal direction.

The gear 80 and pushrod 70 form a drive mechanism with a rack (pushrod 70) and pinion (gear) arrangement. Rotation of the gear 80 by the actuator arm 92 translates into linear movement of the plunger 34 in the distal direction. The gear 80 is sized to provide a precise amount of linear movement of the plunger 34 to deliver a specific quantity of the drug solution 50 upon actuation of the button 90. A spring 108 is connected to the button 90 to bias the button to return to its original extended position and reset the actuator assembly. The spring 108 may be replaced with any suitable compliant member which may be integral with, or connected to the button 90, support 82, or housing 84, for example. The button 90 may be actuated a number of times to deliver any number of drops of the drug solution 50. It is to be understood that other types of actuator assemblies or drive mechanisms may be used without departing from the scope of the invention. For example, a cam and lever arrangement may also be used.

In order to assemble the drug dispenser 10, the cylindrical member 20 is first filled with the drug 22 and the piston 40 is positioned within the member at a location proximal to the connecting passage 36 (FIGS. 2 and 3). The diluent chamber 48 is then filled with the diluent 24 and the plunger 34 is positioned within the cylindrical member 20. The cap 60 is preferably already connected to the cylindrical member 20 to prevent contamination of the drug 22 or escape of the drug from the nozzle 52. The cylindrical member 20 and pushrod 70 are inserted into the support 82 and the housing 84 is snapped into place with the cylindrical member in its non-mix position.

In operation, an axial proximally directed force is applied to the cap 60 to move the cylindrical member 20 to its mix position (FIG. 4). As the cylindrical member 20 moves proximally the length of the diluent chamber 48 is reduced, thus increasing pressure within the chamber (FIGS. 3 and 4). The increase in pressure causes the piston 40 to move distally and open the connecting passage 36. The drug and diluent chambers 44, 48 are now in fluid communication and the diluent 24 flows through the connecting passage 36 to mix with the drug 22 and form the drug solution 50 within the drug chamber. As the cylindrical member 20 moves to its mix position the button 90 moves to its extended position within the window 86 formed in the housing 24. The cap 60 is unscrewed from the cylindrical member 20 in preparation for delivery of the drug solution 50 and the dispenser 10 is positioned over the eye of a person receiving the drug solution (FIG. 5). Upon removal of the cap 60, pressure built up within the cylindrical member 20 is released. The button 90 is pressed radially inward to rotate the gear 80. Upon actuation of the button 90, the arms 92 rotate counterclockwise (as viewed from the right in FIG. 2). The rib 100 extending from the arm 92 engages with the teeth 105 of the ratchet gear 106 and moves the gear 80 counterclockwise. As the gear 80 rotates, the teeth 104 of the gear intermesh with the teeth 76 of the pushrod 70 to move the pushrod and pistons 40, 72 in the distal direction. When the button 90 is released, the spring 108 forces the button to return to its extended (ready) position. Since the teeth 105 only permit movement of the gear 80 by the rib 100 in the counterclockwise direction, the button ratchets clockwise without moving the gear. This prevents the plunger 34 from moving in the proximal direction. The actuator button 90 is now ready to be pushed a second time to deliver another drop of the solution 50. This process may be repeated a number of times.

A safety tab (not shown) may also be incorporated in the drug dispenser 10 to prevent inadvertent actuation of the cylindrical member 20. The safety tab may be formed on the housing 84 or cap 60, for example. A tray configured to hold the drug dispenser 10 in its non-mix position may also be provided with the drug dispenser. The drug dispenser 10 is removed from the tray just prior to use.

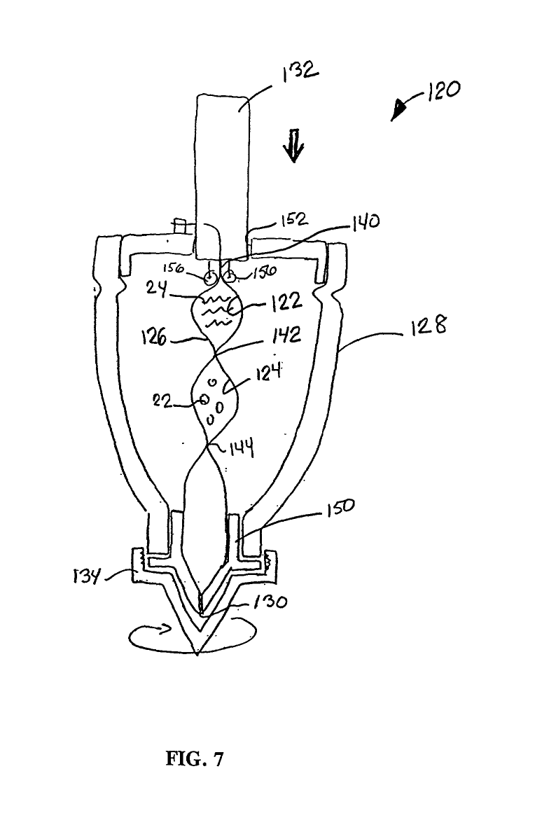

A second embodiment of a drug dispenser is generally indicated at 120 and shown in FIG. 7. The dispenser 120 comprises a sealed diluent chamber 122 and a sealed drug chamber 124 formed in a flexible member (bag) 126. The bag 126 is positioned within a housing 128 which has an opening 130 at a distal end thereof for delivery of the drug solution. A piston 132 is axially movable within the housing 128 to open the sealed chambers 122, 124 and allow the drug 22 and diluent 24 to mix to form the drug solution as further described below. The dispenser 120 further includes a cap 134 which may be threadably engaged with the distal end of the housing 128. The cap 134 may also be attached to the distal end of the housing 128 by a snap detent, or any other suitable means, or the cap 134 may be eliminated.

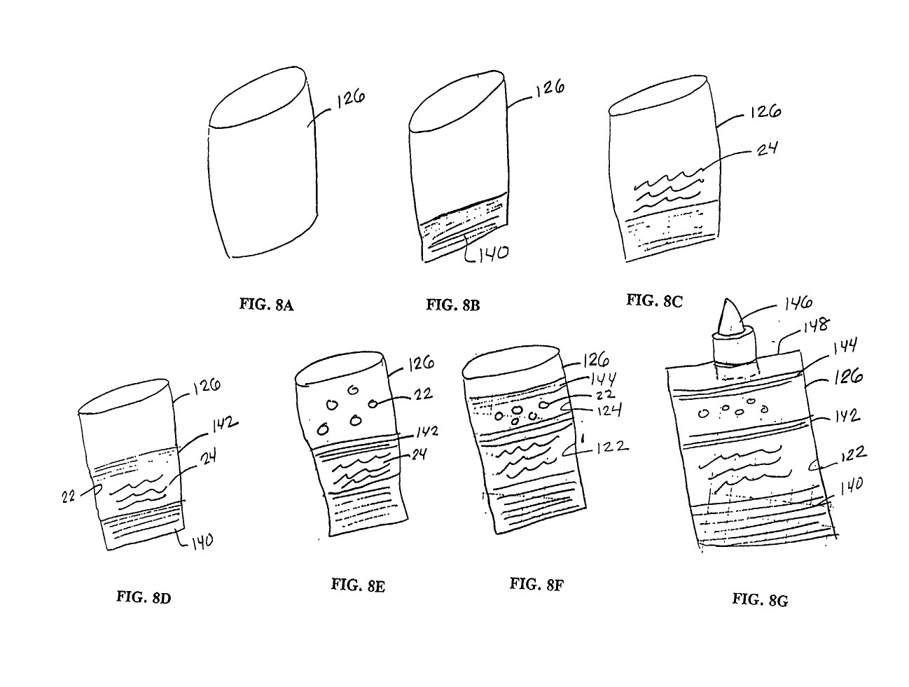

The bag 126 may be formed from any suitable material which is compatible with the drug 22, diluent 24, and drug solution, and capable of being sealed to provide a leak resistance chamber. One method for forming the diluent and drug chambers 122, 124 is shown in FIGS. 8A-8G. The bag 126 initially has two open ends (FIG. 8A). A seal 140 is first formed at the proximal end of the bag (FIG. 8B). The diluent 24 is then poured into the bag 126 to fill approximately one-third of the bag (FIG. 8C). A seal 142 is formed above the diluent 24 to form the diluent chamber 122 (FIG. 8D). The drug 22 is next poured into the bag 126 and a third seal 144 is formed to create the drug chamber 124 (FIGS. 8E and 8F). A nozzle 146 is then attached to a distal end 148 of the bag and the bag is inserted into the housing 128 (FIGS. 7 and 8G). The seals 140, 142, 144 may be formed by heat sealing the bag 126, or with adhesives, ultrasonic, or solvent bonding, or any other suitable method. The volume of the diluent chamber 122 and drug chamber 124 may vary to accommodate different ratios of diluent 24 and drug 22.

As shown in FIG. 7, the housing 128 has a retainer 150 at its distal end for holding the bag 126 within the housing, and an opening 152 at its proximal end for receiving the piston 132. The nozzle 146 may be configured for attachment to the housing to eliminate the retainer 150. The opening 152 is formed in the proximal end of the housing 128 which acts as a guide for the piston 132. A pair of rollers 156 extends from a distal end of the piston 132. The rollers 156 are positioned on opposite sides of the bag 126 and are spaced apart a distance sufficient to squeeze the chambers 122, 124 as the piston is moved distally past the chambers to individually pressurize the chambers and break the seal 142 between the chambers and the seal 144 at the distal end of the drug chamber 124. As the rollers 156 move past the diluent chamber 122 they squeeze the flexible chamber, thus increasing the pressure within the chamber and causing the seal 142 between the diluent chamber and drug chamber 124 to open and form a mixing chamber. The seal 140 located 126 should be stronger than the seal 142 located between the diluent and drug chambers 122, 124 so that upon pressurization of the diluent chamber, the seal 142 breaks open with the seal 140 staying intact. As the piston 132 continues to move in the distal direction, the seal 144 located distally of the drug chamber 124 is broken and the drug dispenser 120 is ready for delivery of the drug solution from the opening 130 after removal of the cap 134.

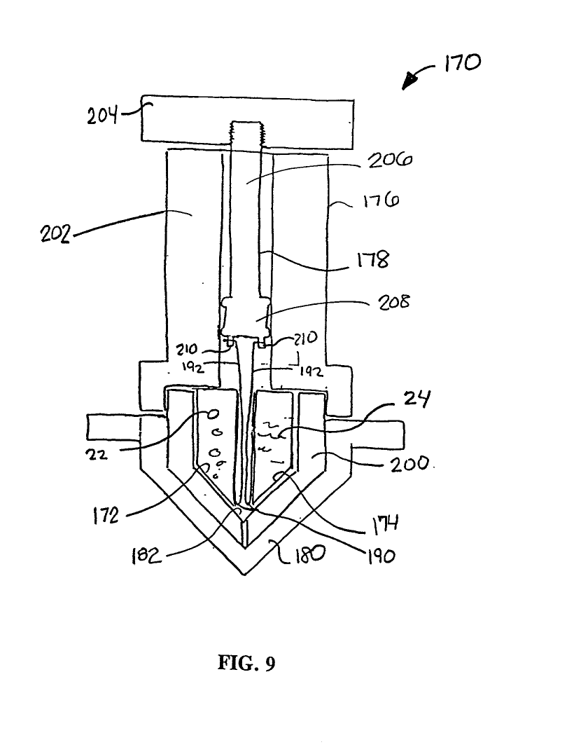

A third embodiment of the present invention is generally indicated at 170 and shown in FIG. 9. The dispenser 170 comprises a sealed drug chamber 172, a sealed diluent chamber 174, a housing 176 for containing the sealed chambers, and a piston 178 axially movable within the housing. The piston 178 is configured to open the sealed chambers 172, 174 as it moves in the proximal direction to permit the drug 22 and diluent 24 to mix and form the drug solution. A cap 180 is provided for sealing a distal end of the housing 176 which forms a mixing chamber 182. Since the chambers 172, 174 are sealed prior to mixing, the cap 180 may be eliminated.

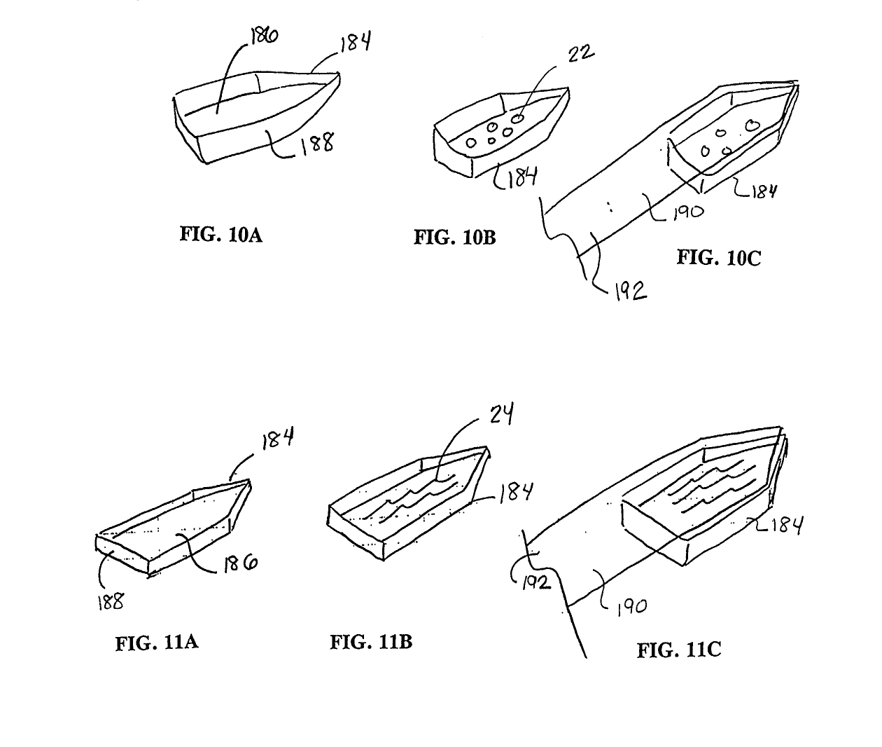

The drug and diluent chambers 172, 174 are each formed from a container 184 having a bottom wall 186, a sidewall 188, and an open top (FIGS. 10A and 11A). It should be understood that the shape of the container 184 may be different than the one shown without departing from the scope of the invention. The drug container may also have a different shape or size than the diluent container. The drug 22 and diluent 24 are each inserted into their respective containers 184 (FIGS. 10B and 11B). Each container 184 is then sealed with a flexible cover sheet 190 (FIGS. 10C and 11C). The sheet 190 is sized to cover the container 184 and have an end portion 192 which extends beyond the length of the container for attachment to the piston 178 as further described below. The sheet 190 may be formed from aluminum foil or any other suitable material.

The housing 176 comprises a retainer 200 at its distal end for receiving the drug and diluent chambers 172, 174 and a guide 202 for guiding the piston 178. The drug and diluent chambers 172, 174 are positioned within the retainer 200 and upon opening the chambers, the drug 22 and diluent 24 are mixed within the retainer 200 which forms the mixing chamber 182. The piston 178 includes a flange 204 at is proximal end for actuation of the piston, a rod 206 threadably engaged with the flange and a piston head 208. The rod 206 may also be attached to the flange 204 by adhesive or any other suitable means. The distal end of the piston head 208 includes two clips 210 for receiving the cover sheets 190. The clips 208 may be spring biased to hold the sheets 190, for example. The clips 210 may also be eliminated and the sheets 190 attached directly to the piston head 208 by glue, for example.

In order to assemble the dispenser 170, the drug and diluent containers 184 are filed with the drug 22 and diluent 24, respectively, and sealed (FIGS. 10C and 11A-11C). Each container 184 is then inserted into the retainer 200 and the flexible covers 190 are attached to the distal end of the piston head 208 (FIG. 9). The guide 202 is then positioned over the piston 178 and attached to the retainer 200. The flange 204 is threaded onto the proximal end of the piston 178. The drug 22 and diluent 24 are mixed just prior to delivery of the drug solution by pulling the piston 178 in the proximal direction. The piston 178 pulls the sheets 190 at least partially off the containers 184 and the drug 22 and diluent 24 are mixed within the mixing chamber 182. Various actuation devices, as are well known by those skilled in the art, may be used with the drug dispenser 170 to control delivery of the drug solution from the dispenser. For example, the piston may be a reciprocating piston which advances the plunger one way in controlled amounts.

The drug dispensers 10, 120, 170 described above are preferably used for ophthalmic delivery of a drug solution. It is to be understood that the dispensers 10, 120, 170 may also be used to deliver the drug solution by injection, inhalation, or any other suitable method.

Preferably the drug 22 is a lyophilized drug in the form of spheres or beads prepared as described in copending commonly owned U.S. patent application Ser. No. 08/965,660, entitled Stabilized, Dry Pharmaceutical Compositions for Drug Delivery and Methods of Preparing the Same, the disclosure of which is incorporated herein by reference. In that process the drug is dissolved in a solvent, such as water, along with fillers, such as polyethylene glycol, myo-inositol, polyvinylpyrrolidone, bovine serum albumin, dextrin, mannitol, trehalose, sodium carbonate, sodium bicarbonate, boric acid and its salts, dextrose, sodium acetate, sodium or potassium phosphates, polyvinyl alcohol-polyvinyl acetate copolymers, and the like. These fillers are used alone or in combination. Surfactants, such as Triton X-100®, sodium laurel sulfate, cetyl trimethyl ammonium chloride, and the like, may be added. Separate buffer components may also be added, if required. Preservatives may also be included in the formulation if the reconstituted solution is to be stored for any appreciable amount of time.

The drug and the filler(s) along with buffer components and surfactants, if desired, are dissolved to prepare an essentially homogeneous solution. The term homogeneous should not be interpreted to imply that colloids or micelles might not exist in the liquid phase. Colloids, micelles, and similar materials can exist as suspensions that behave mechanically as true solutions as is well known in the colloid chemistry art. The resulting solution may optionally be degassed prior to dispensing and is dispensed as precisely measured droplets. The droplet size is typically from about 1.5 to about 20 microliters. This process will typically produce dry beads ranging from about 1 mm to about 4 mm in diameter depending upon the solid content of the dispensed solution, its chemical composition, and the method used to dry the solid.

Droplets are produced by pumping the solution using a precise pump, usually of a direct displacement type, through an appropriate nozzle. The nozzle tip is typically tapered and has a wall thickness generally ranging from about 0.005 inches to about 0.020 inches depending upon the properties of the solution being dispensed. Pumps such as an IVEK model AAA pump (N. Springfield, Vt.) are particularly suitable for this use. The solution is dispensed with a drop rate of from about 1 to about 3 drops per second. There is no lower limit to drop frequency and the upper limit is determined by the rate of solidification of the dispensed material. The dispensed droplets fall into a liquid bath that causes the droplet to form into a solid sphere. The mechanism of sphere formation may be freezing, solvent incompatibility or chemical reaction or combinations thereof. In a preferred embodiment, spheres are formed by freezing which is accomplished by allowing the droplet to fall into a bath of liquid nitrogen. This method is used primarily to produce spheres that dissolve immediately since the freezing step is followed by a lyophilization step. Lyophilization produces spheres with low density. In other words, the solid mass has a large void volume.

Suitable drugs for use in the invention dispensers include, but are not limited to, pharmaceuticals and peptide and polypeptide drugs such as glucagon, insulin, oxytocin, thyrotrophin releasing hormone (TRH), leucine-enkephalin, methionine-enkephalin, somatotropin, oxytocin, vasopressin, lypressin, alphaneoendorphin, beta-neoendorphin, luteinizing hormone releasing hormone (LHRH), dynorphin A, dynorphin B, somatostatin, secretin, calcitonin, ACTH, growth hormone releasing hormone, concanavalin, ribonuclease, lysozyme, ribonuclease, beta-lipotropin, gamma-lipotropin, and the like.

It is to be understood that the drug dispensers 10, 120, 170 may be used to deliver a drug solution formed from a solid and liquid different than those described herein, or from two different liquids.

In view of the above, it will be seen that the several objects of the invention are achieved and other advantageous results attained.

As various changes could be made in the above constructions and methods without departing from the scope of the invention, it is intended that all matter contained in the above description and shown in the accompanying drawings shall be interpreted as illustrative and not in a limiting sense.

What is claimed is:

1. A drug dispenser for administering a drug solution ophthalmically, the dispenser comprising:
a cylindrical member having an open proximal end, a distal end having a hole formed therein for dispensing the drug solution, and a connecting passage formed in a sidewall thereof;
a plunger receivable in the proximal end of the cylindrical member, the cylindrical member being axially slidable on the plunger between a non-mix position and a mix position;
a piston having a distal end and a proximal end and slidably disposed within the cylindrical member in sealing engagement therewith, the distal end of the piston being located proximally of the connecting passage and forming a drug chamber with the distal end of the cylindrical member for storing a drug when the cylindrical member is in its non-mix position, the proximal end of the piston being distally spaced from the plunger to form a diluent chamber therebetween for storing a physiologically acceptable liquid diluent when the cylindrical member is in its non-mix position;
wherein when the cylindrical member is moved to its mix position the drug chamber and diluent chamber are in fluid communication and the diluent is free to flow through the connecting passage to mix with the drug to form the drug solution.

2. The dispenser of claim 1 wherein the plunger comprises a pushrod and a sealing piston located at the distal end of the rod.

3. The dispenser of claim 1 further comprising a cap configured for receiving the distal end of the cylindrical member.

4. The dispenser of claim 3 wherein the cap comprises a seal for sealing the hole in the distal end of the cylindrical member.

5. The dispenser of claim 1 further comprising a housing at least partially enclosing the cylindrical member.

6. The dispenser of claim 1 further comprising an actuator assembly for moving the plunger in the distal direction to force the drug solution out of the hole in the distal end of the cylindrical member when the cylindrical member is in its mix position.

7. The dispenser of claim 6 wherein the actuator assembly comprises an actuation device configured for engagement with the plunger for moving the plunger in only the distal direction.

8. The dispenser of claim 7 wherein the actuation device and plunger each comprise intermeshing teeth which allow limited movement of the plunger upon actuation of the actuator assembly.

9. The dispenser of claim 6 wherein the actuator assembly includes an actuation button operable only when the cylindrical member is in its mix position.

10. A drug dispenser for administering a drug solution, the dispenser comprising:
a sealed diluent chamber containing a physiologically acceptable liquid diluent, at least a portion of the chamber being flexible;
a sealed drug chamber containing a drug, at least a portion of the chamber being flexible;
a housing containing the diluent chamber and the drug chamber and having an opening at a distal end thereof for delivery of the drug solution; and
a piston axially movable within the housing to open the sealed chambers and allow the drug and diluent to mix to form the drug solution.

11. The dispenser of claim 10 further comprising a bag forming the diluent chamber and the drug chamber, the bag having a seal to separate the chambers.

12. The dispenser of claim 11 wherein the bag further comprises a third chamber having an opening formed therein for delivery of the drug solution, and a second seal between the third chamber and one of the diluent chamber and drug chamber.

13. The dispenser of claim 11 wherein the seal is heat sealed.

14. The dispenser of claim 11 further comprising a pair of rollers extending from a distal end of the piston, the rollers being located on opposite sides of the bag and spaced apart a distance sufficient to squeeze one of the chambers as the piston is moved distally past the chamber to pressurize the chamber and break the seal to allow the diluent and drug to mix.

15. The dispenser of claim 10 wherein each chamber comprises a container having an opening formed therein and a flexible sheet covering the opening.

16. The dispenser of claim 15 wherein the flexible sheet comprises foil.

17. The dispenser of claim 15 wherein each sheet is connected to a distal end of the piston so that movement of the piston in the proximal direction removes each sheet from its respective container to allow the drug to mix with the diluent to form the drug solution.

18. The dispenser of claim 17 wherein movement of the piston in the distal direction forces the drug solution out from the opening in the distal end of the housing.

19. A drug dispenser for administering a drug solution formed from a drug and diluent, the dispenser being movable between a non-mix position wherein the drug and diluent are separated and a mix position wherein the drug and diluent are mixed to form the drug solution, the drug dispenser comprising a piston and an actuator assembly for delivering the drug when the dispenser is in its mix position, the actuator assembly comprising a drive mechanism operable to move the piston in a distal direction and a ratchet gear for preventing movement of the piston in a proximal direction.

20. The dispenser of claim 19 wherein the actuator assembly further comprises an actuation button operable only when the dispenser is in its mix position.

21. The dispenser of claim 20 wherein the button is spring biased to an extended position to allow repeated actuation of the actuator assembly.

22. The dispenser of claim 19 wherein the drive mechanism comprises a rack and pinion.
